SEQUENCE LISTING (2 sequences)

SEQ ID NO: 1 - DNA, 8 bp, Rattus sp.
atatggcc

SEQ ID NO: 2 - DNA, 16 bp, Rattus sp.
gagaatctaggctcat

(19) United States
(12) Patent Application Publication
(10) Pub. No.: US 2021/0092942 A1
(43) Pub. Date: Apr. 1, 2021

(54) NOVEL IMMUNODEFICIENT RAT FOR MODELING HUMAN CANCER

(71) Applicant: Hera Testing Laboratories, Inc., Lexington, KY (US)

(72) Inventors: John Stuart CRAWFORD, Lexington, KY (US); Tseten YESHI, Lexington, KY (US); Fallon NOTO, Lexington, KY (US); Goutham NARLA, Pepper Pike, OH (US)

(21) Appl. No.: 16/499,439

(22) PCT Filed: Mar. 30, 2018
(86) PCT No.: PCT/US2018/025368

§ 371 Date: Sep. 30, 2019

Related U.S. Application Data
(60) Provisional application No. 62/479,857, filed on Mar. 31, 2017.

(51) Int. Cl. A01K 67/027; C07K 14/47; C07K 14/715

(52) U.S. Cl. CPC A01K 67/0276; C07K 14/4705; C07K 14/7155; A01K 2207/12; A01K 2267/0331; A01K 2217/075; A01K 2217/15; A01K 2227/105; A01K 2207/15

(57) ABSTRACT

Disclosed herein are methods and compositions for performing assays for determining efficacy of drugs using rat SCID models that exhibit excellent take rates, and excellent tumor growth rates. The methods and compositions offer dramatically improved efficiencies compared to corresponding mouse equivalents.

### CROSS REFERENCE TO RELATED APPLICATION

This application claims priority to U.S. Prov. No. 62/479,857 filed Mar. 31, 2017, the disclosure of which is hereby incorporated by reference for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: Hera sequence listing_ST25.TXT, date recorded: Mar. 26, 2018, file size 4 kilobytes).

### BACKGROUND

Animal models of human cancer offer the potential to study human tumor growth kinetics, genetic variance among human cancers, and provide in vivo platforms for drug efficacy testing. Immunodeficient mouse models have been invaluable in modeling a wide range of human cancers and testing drug efficacy. However, the use of mouse models is limited by the lack of growth of many cancer cell lines in mice, the variability of growth kinetics and take rates from mouse to mouse. Drug efficacy studies are difficult due to the limited number of cell line-based models to test novel agents, the large sample sizes needed to power mouse in vivo studies, and the small tumor size and lack of ability to perform serial sampling of tumor and blood for pharmacodynamic/pharmacokinetic studies.

These challenges also occur in patient derived xenograft (PDX) models which are based on the transfer of primary tumors directly from the patient into an immunodeficient mouse, in which take rates are even lower and growth rates slower to obtain sufficient numbers of tumors for drug efficacy studies. PDX models have shown promise as clinical diagnostics to determine if a particular therapeutic regimen will be efficacious in a specific patient. However, PDX models are not routinely used because mouse hosts of PDX models can suffer from long latency periods after engraftment and variable engraftment rates (also referred to as “take rates”). Tumor graft latency, measured as the time between implantation and the development of a progressively growing xenograft tumor can range from two to twelve months (Siolas et al. Cancer Research 2013). In mice, the engraftment phase and expansion phase are often too long for the efficacy study to take place before the treatment of the patient must occur.

There is therefore a pressing need for models that provide improved take rates as well as growth rates for drug efficacy studies.

### BRIEF DESCRIPTION OF THE DRAWING

FIG. 1A-1C. Analysis of immune cell populations in a Rag2 KO rat. FIG. 1A) Rag2 KO rat thymocytes contain fewer mature T cells (bottom panel), compared to a wild-type control (top panel). Insets show thymus with organ weight. The majority of thymocytes are CD4- and CD8-double negative in a Rag2 KO rat. FIG. 1B) The spleen contains no mature B cells as demonstrated by lack of CD45R (B220)+/IgM+ cells (bottom panel) compared to wild type spleen (top panel). FIG. 1C) The Rag2 KO rat spleen has an increased NK cell population (bottom panel) compared to the wild-type (top panel). Whereas the wild-type rat has 3.97% NK cells in the splenocytes and less than 1% NK cells in the thymocyte population, the Rag2 KO rat splenocytes and thymocytes contained 43.94% and 5.41% NK cells, respectively.

FIG. 2. Immunophenotyping of a I2rg;Rag2 KO rat. A-C: immunophenotyping of thymocytes and splenocytes. Wild-type, left panels; Il2rg;Rag2KO rat (also referred to as “Rag2^{-/-}, IL2rg^{-/-}”), right panels. FIG. 2A) CD4+/CD8+ mature T cells are absent from Il2rg;Rag2 KO rat thymocytes, compared to a wild-type control. The lack of thymus tissue in the Il2rg;Rag2 KO rat results in a low recovery of viable thymocytes. FIG. 2B) The Il2rg;Rag2 KO rat spleen contains no mature B cells as demonstrated by lack of CD45R (B220)+/IgM+ cells, compared to a wild-type spleen. Percentages are provided as % of total PBMCs. FIG. 2C) NK cells in the ILR2g and Rag2 KO rat spleen are similar to or less than the amount of NK cells in the wild-type rat. The Rag2^{-/-}, IL2rg^{-/-} rat spleen has slightly lower NK cells compared to the wild-type spleen (2.81% vs. 3.96%, respectively). Strikingly, circulating NK cells in the peripheral blood of the SRG rat are significantly reduced (0.5%) relatively to wild-type levels (10.1%). NK cells are measured by CD161 antibody. Percentages are provided as % of total PBMCs. FIG. 2D-F illustrate immunophenotyping of peripheral blood. Wild-type, left panels; double KO rat, right panels. FIG. 2D) T cells are significantly reduced in peripheral blood of the double KO rat (1.6% CD4+, 5.3% CD8+, 1.2% CD4+/CD8+) compared to wild-type rat (37.4% CD4+, 36.6% CD8+, 3.5% CD4+/CD8+). Percentages are provided as % of total PBMCs. FIG. 2E) The I2rg;Rag2 rat is completely devoid of circulating mature B cells compared to wild-type animals. Percentages are provided as % of total PBMCs. FIG. 2F) The Rag2^{-/-}, IL2rg^{-/-} rat has significantly reduced circulating NK cells (0.5% CD161a+) compared to wild-type rat (10.1% CD161a+). FIG. 2G shows reduced NK cells in the Il2rg;Rag2 rat versus the Nude rat.

FIG. 3: Immunodeficient rat and mouse model immune phenotype summary table. Immune phenotype data for NOD.CB17-Prkdc^{scid}, commonly called “NOD scid” or “SCID” mice demonstrates a lack of mature B- and T-cells and reduced NK-cell activity (Physiological Data Summary—NOD.CB17-Prkdcscid/J (001303) JAX.org). Immune phenotype data for NOD.Cg-Prkdc&^{scid}Il2rg^{tm1Wjl}/SzJ, a double knock out for Prkdc and Il2rg (NSG™ mice; Jackson Laboratories). This is a mouse equivalent of the ILR2g and Rag2 KO rat and, like the rat, demonstrates a lack of mature T, B, and functional NK cells (Physiological Data Summary—NOD.Cg-Prkdc^{scid}Il2rgtm1Wjl/SzJ (005557) JAX.org). Immune phenotype data for NU/J, commonly called Nude mice demonstrates a lack of T cells and a partial defect in B cell development (Physiological Data Summary—NU/J (002019) JAX.org).

FIG. 4: Enhanced survival of non-small cell lung cancer (NSCLC) cell line H358 and tumor kinetics in the Rag2 KO rat compared to NSG and Nude mice. H358 cancer cells growth in the Rag2 KO rat compared to the nude (nu/nu) and NSG™ mice. H358 cancer cells were transplanted subcutaneously in the Rag2 KO rat. Three groups of 6 rats received either 1×10⁶, 5×10⁶, or 10×10⁶ cells in 5 mg/ml Geltrex®. Growth rate was directly proportional to the amount of cells transplanted. These data are displayed in conjunction with data showing tumor growth kinetics of the H358 cell line in Nude and NSG™ mice, both of which were transplanted with 10e6 cells subcutaneously.

FIGS. 5A-5E illustrates various studies comparing xenograft growth. FIG. 5A shows Raw Data: Tumor Weight Reports, Body Weight Reports, Animal Fate Report, Clinical Observations Data Report, Post-Study Collections Inventory for VCaP implantation 5×10e6 cells/mouse subcutaneous right flank in 50% Matrigel/50% Media in 0.1 ml in NOD and ICR SCID mice. FIG. 5B shows the data in graph form for the ISC SCID mouse. FIG. 5C shows the data in graph form for the NOD SCID mouse. FIG. 5D shows the data for the Rag2^{-/-}, IL2rg^{-/-} SCID rat. Each line shows a different animal and illustrates the take rate and growth.

FIG. 6: Establishment and testing of PDX models. Tumor specimens are obtained from the consented patients and introduced to a rat, referred to as passage 0 or P0. Non-necrotic areas of these tumors are sectioned into ˜2-3 mms pieces and, after processing, implanted subcutaneously into anaesthetized SCID rats. During the engraftment phase, tumors are allowed to establish and grow and then are harvested upon reaching a size of). Similar protocols are employed for subsequent expansion passages (P2 . . . PN). Typically in mice, biological assays are performed on tumors in early generations but are not available for studies as early as P1 or P2 passages. In contrast, the SCID rat models disclosed herein provide xenograft models with rapid growth that are available for studies at the P1 and P2 stages.

FIGS. 7A-7C illustrate the difference in growth rate of the NSG mouse versus the Rag2^{-/-}, IL2rg^{-/-} rat with HCT-116 xenografts. FIG. 7A shows tumor volume in the NSG mouse. FIG. 7B shows tumor volume in the Rag2^{-/-}, IL2rg^{-/-} rat. Equal numbers of cells were introduced in both cases. These data illustrate that the growth rate and ultimate tumor volume is remarkably improved in the Rag2^{-/-}, IL2rg^{-/-} rat.

FIGS. 8A-8E illustrate humanization of the Rag2^{-/-}, IL2rg^{-/-} rat using human PBMCs as a xenograft. FIG. 8A illustrates the growth and propagation of CD45+ human cells in the rat. FIG. 8B shows the percentage of those CD45+ cells that are CD3+ T-cells. FIG. 8C shows human Tn cell (hCD4+) and human CTLs (hCD8+) over time. Dual positive cells are also shown. FIG. 8D shows a FACS analysis of the cells comparing hCD4+ and hCD8+ T cells. FIG. 8E shows human B cells (hCD20)

FIGS. 9A-9B illustrate improved growth kinetics of a NSCLC primary tumor taken from a biopsy in the Rag2^{-/-}, IL2rg^{-/-} rat versus the NSG mouse (Prkdc^{scid} Il2rg^{tm1Wjl}/SzJ). FIG. 9A shows growth in the rat. FIG. 9B shows growth in the mouse.

FIGS. 10A-10B illustrate improved growth kinetics of an ovarianprimary tumor taken from a biopsy in the Rag2^{-/-}, IL2rg^{-/-} rat versus the NSG mouse (Prkdc^{scid Il}2rg^{tm1Wjl}/SzJ). FIG. 10A shows growth in the rat. FIG. 10B shows growth in the mouse.

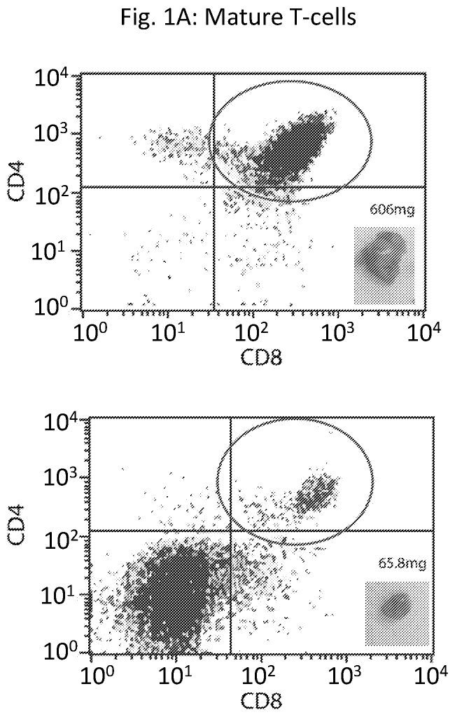

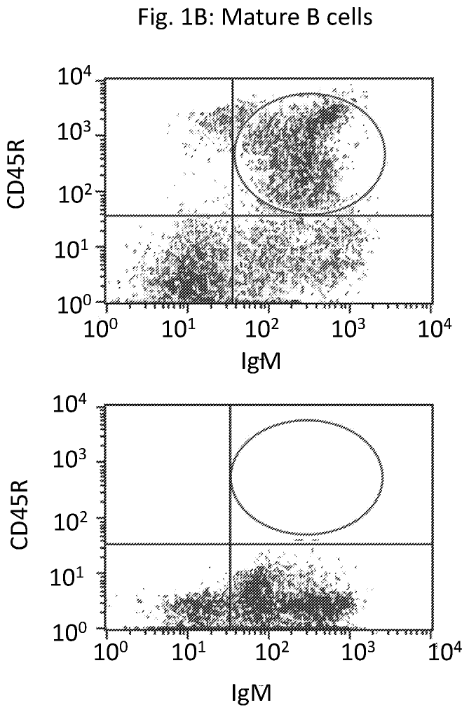

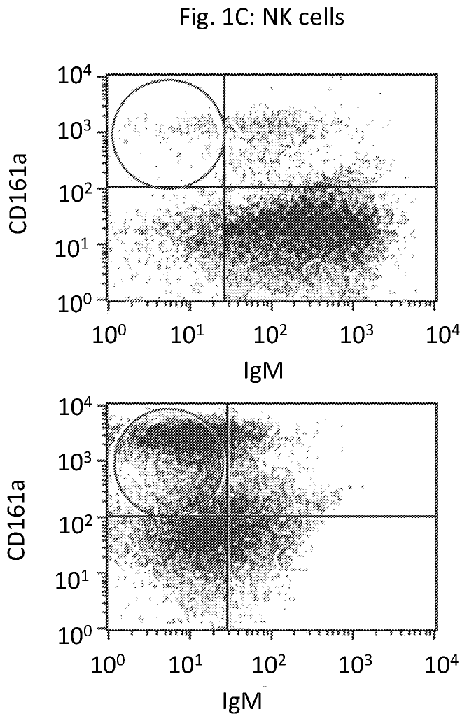

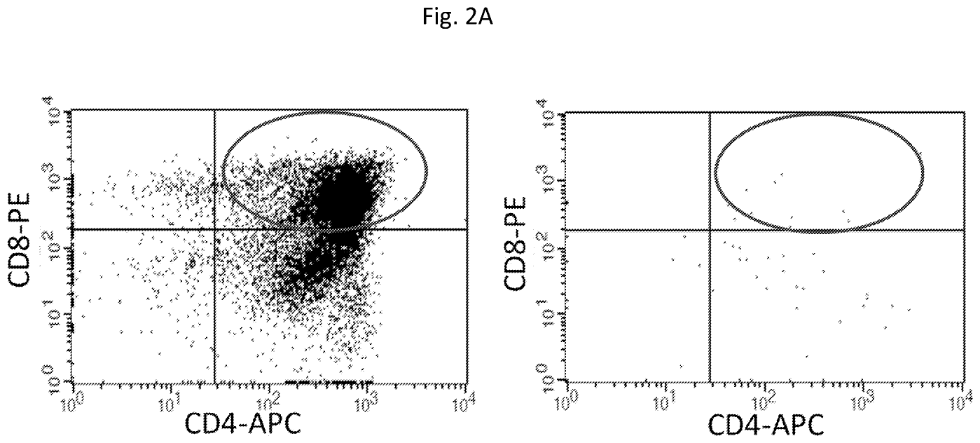

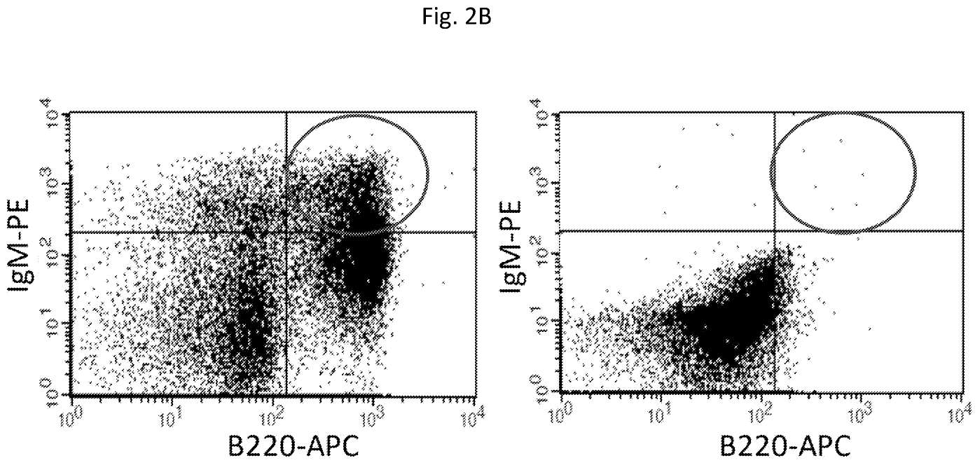

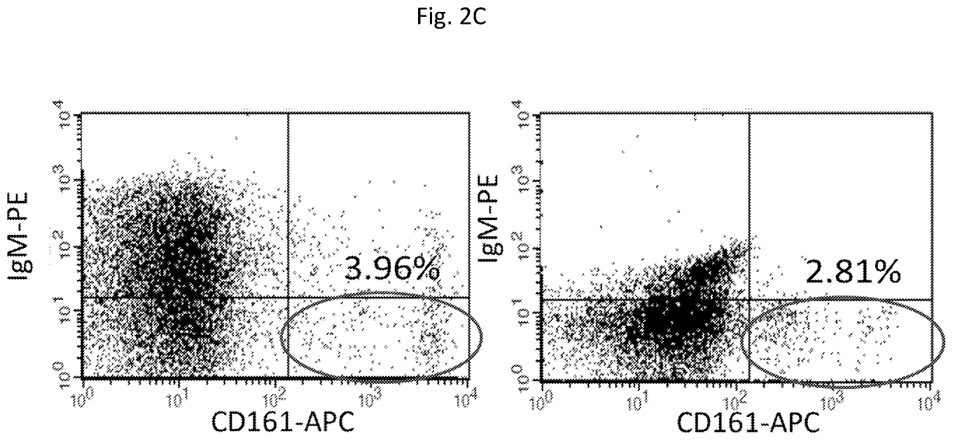

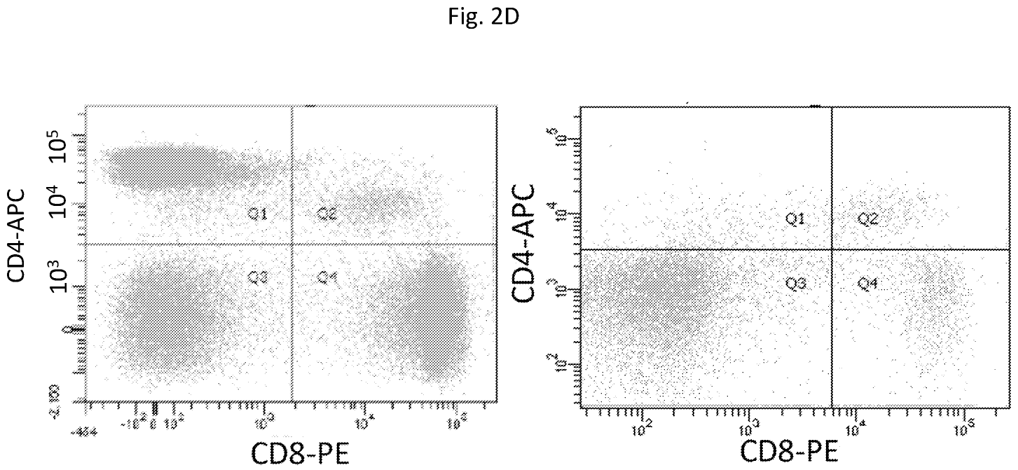

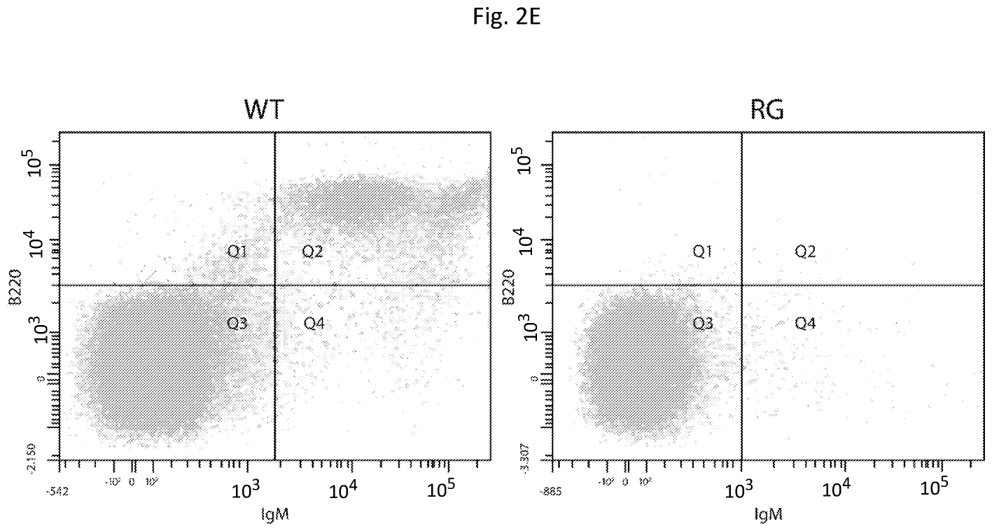

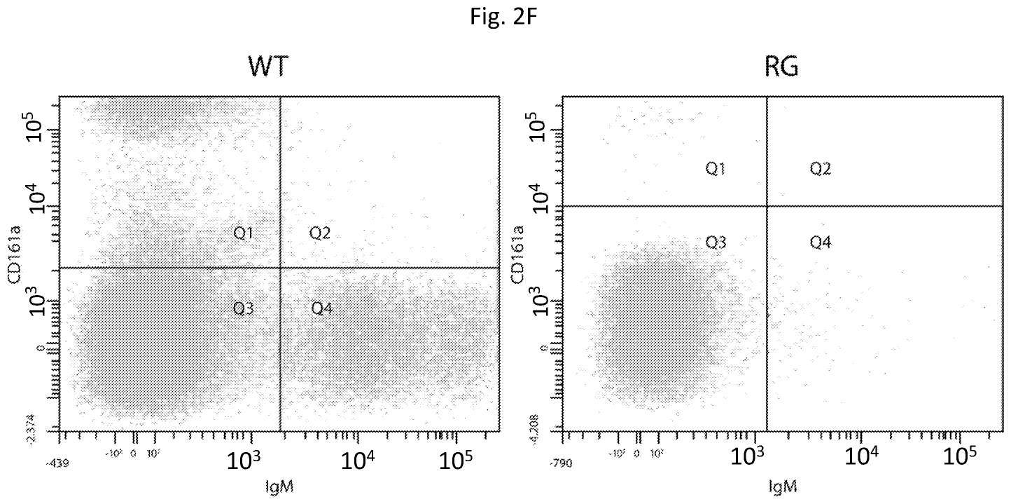

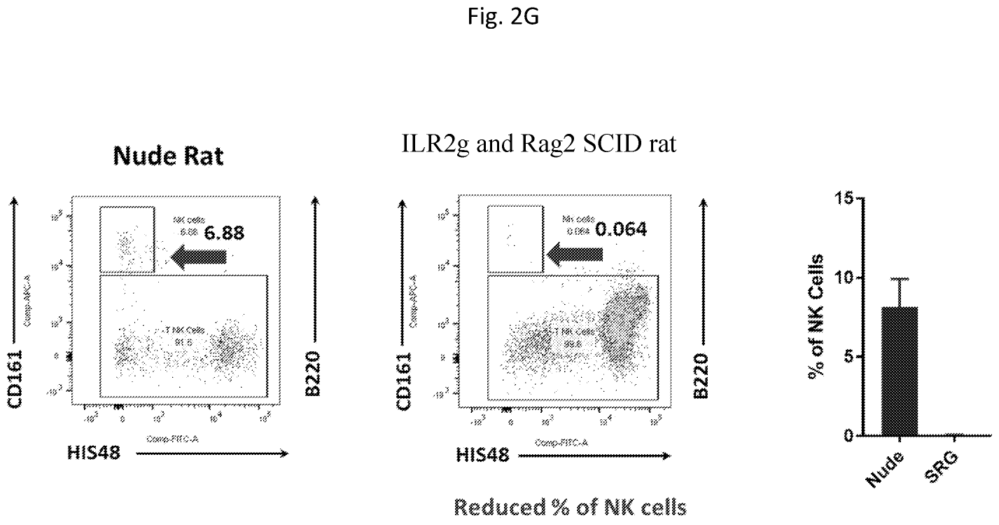

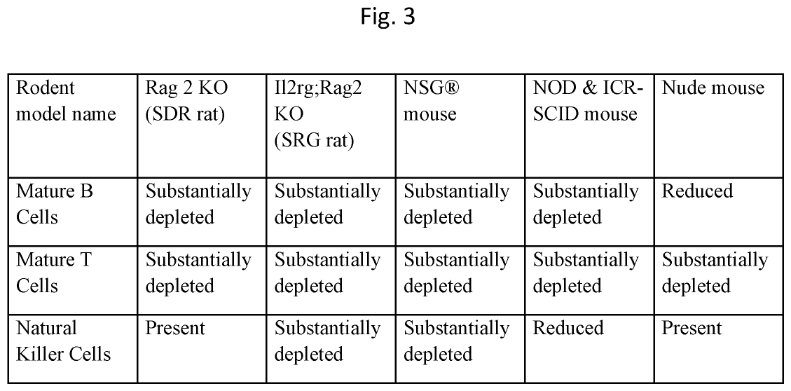

### SUMMARY OF THE INVENTION

Disclosed herein are methods of screening a drug for treating a tumor comprising (a) administering the drug to a Severe Combined Immune Deficiency (SCID) rat having a xenograft tumor; wherein the SCID rat is a knockout rat comprising one or more genetic mutations that result in substantially depleted B-cells, T-cells and NK-cells. The effect of the drug on the tumor may then be determined. The SCID rat containing the xenograft tumor may be an F1 phase or an F2 passage. Further, the SCID rat may have a percentage take rate at least 10 points higher than a corresponding SCID mouse. The SCID rats disclosed herein may also exhibit tumor growth rate at least 5%, or more, than a corresponding SCID mouse. In particular aspects, the methods may use a SCID rat containing a deletion IL2Rg and the Rag2 genes; for example, the Rag2 deletion may comprise, or consist of, SEQ ID NO: 1, and the IL2Rg gene deletion may comprise, or consist of, SEQ ID NO:2.

Methods for performing drug efficacy assays using patient derived xenografts are also provided. The methods involve introduce introducing a patient derived xenograft, such as an ovarian cancer or a non-small cell lung cancer (NSCLC) into a Severe Combined Immune Deficiency (SCID) rat having substantially depleted mature B-cells, T-cells and NK-cells, and administering a drug to the rat. The rat may be a P1 or P2 passage rat. In addition to the methods disclosed herein, a Severe Combined Immune Deficiency (SCID) rat having a xenograft tumor for use in performing drug efficacy assays is provided.

### DETAILED DESCRIPTION

As used herein, the term “take rate” refers to the percentage of animals in which a xenograft is found to grow.

As used herein, the term “about” refers to a value plus or minus 10% of the indicated numerical value.

As used herein, “coding sequence” refers to a nucleic acid, for example DNA, which, when expressed, results in the production of that RNA, polypeptide, protein, or enzyme. Coding sequence for a protein encompass a start codon (usually ATG) and a stop codon. Regulatory sequences that are involved in controlling expression of the coding sequence are outside of the coding sequence but remain part of the gene.

As used herein “deletion,” in the context of mutation, means a type of mutation that involves the removal of genetic material, which may be one or more nucleotides in the gene, including either the coding sequence of the regulatory sequences. Deletion may result in reduced or eliminated expression of the protein. In other aspects, the protein may be expressed but may have an altered sequence such that the protein no longer functions.

As used herein, “genetically modified” means a gene altered from its native state (e.g., by insertion mutation, deletion mutation), or that a gene product is altered from its natural state, using recombinant DNA techniques.

As used herein, “drug,” as used in the context of for example, drug efficacy or screening assays, encompasses both pharmaceutical-type drugs and biologic-type drugs such as antibodies.

As used herein, “knock-out” means a mutation in a gene of an animal, typically a rat, that reduces the biological activity of the polypeptide normally encoded by the gene by at least 80% compared to the unaltered gene. The mutation may be, for example, an insertion or a deletion resulting in frameshift mutation or missense mutation. Typically, the mutation is a deletion.

As used herein, the term “SCID” (Severe Combined Immune Deficiency) in the context of an animal refers to an animal having genetic mutations that result in depleted or substantially depleted B-cells, T-cells and NK cells. The animals typically are knock out animals with the result that that the proteins are not expressed at all, are expressed at such a low level that the protein does not support the normal biological function of the protein, or that the expressed protein is mutated with respect to the wild-type protein such that it does not support the normal biological function of the protein. In particular aspects, the SCID animal is a knock out rat with a mutation in both the Rag2 and Il2rg genes. In other aspects, the animal is a knock out animal with a mutation in both the Prkdc and Il2rg genes.

As used herein, a “corresponding” animal is an animal having the same functional differences, relative to the wild-type animal. For example, a SCID knock out mouse having mutated Prkdc and Il2rg genes may be a corresponding animal for a SCID rat having mutated Prkdc and Il2rg and may also be a corresponding animal for a SCID rat having mutated Rag2 and Il2rg genes. In each cases, the mutations in a corresponding animal results in a phenotype that is substantially identical to that in the animal from the other species.

As used herein, the term “substantially” refers to circumstance which is almost complete. For example, if a particular cell type is substantially depleted, only a residual amount of that type remains and is unable to support normal cell function. For example, if a cell type is substantially depleted, the amount of depletion is, compared to a normal circumstance, (e.g. wild type animal lacking identified mutations) decreased by at least 80%, at least 90%, at least 95%, or at least 99%, unless otherwise specified.

Disclosed herein are compositions and methods for producing transgenic rat models having improved engraftment of xenografts. The transgenic rat models are SCID rats. In particular, the rats are depleted or substantially depleted with respect to B-cells, T-cell, and NK cells. In particular, disclosed herein for the first time is a homozygous Rag2, Il2rg double knockout SCID rat model (Rag2^{-/-}, IL2rg^{-/-}) on the Sprague-Dawley strain as a competent host for human cancer cell lines and efficacy studies as well as human PBMCs for immune system humanization. In contrast to the corresponding mouse, the Rag2^{-/-}, IL2rg^{-/-} rat is a valuable in vivo human tumor model with the potential for immuno-oncology studies.

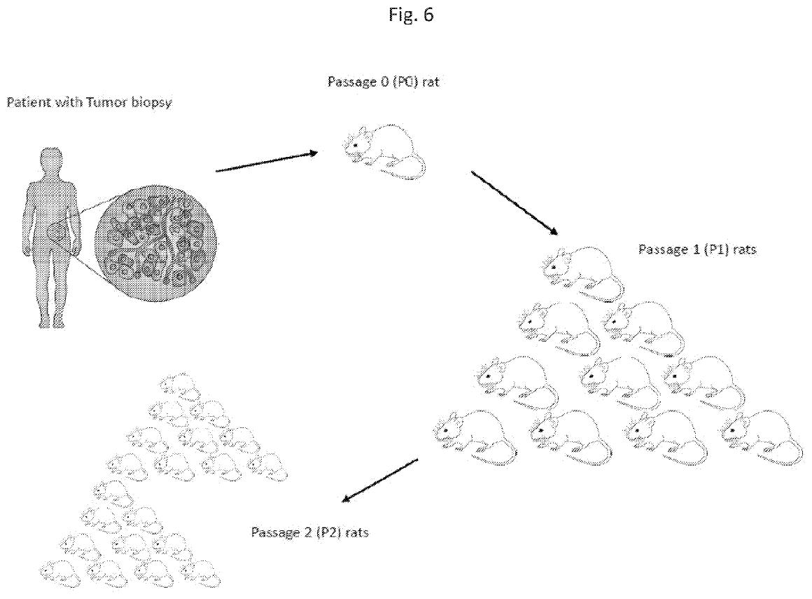

The SCID rat hosts of PDX models with improved engraftment efficiency and faster growth kinetics (engraftment and expansion phases from FIG. 6) as well as the ability to grow larger tumors provides a solution for the problems PDX models suffer when hosted in mice. Applicant has characterized two immunodeficient rat models, one with a functional deletion of the Rag2 gene (SDR™ rat; HeraBioLabs, Inc., Lexington Ky.) and another with a functional deletion in both the Rag2 and I2rg genes. In certain aspects, the ILR2g and Rag2 KO rat is an SRG™ rat from HeraBioLabs, Inc., Lexington Ky. Improved cancer cell line engraftment rate, tumor growth kinetics were observed in the immunodeficient rat models in comparison with state-of-the-art mouse models, NSG® and Nude. The immunodeficient rat models do not have significantly different immune phenotypes. Both the SCID rat and the corresponding NSG SCID mouse are depleted or substantially depleted with respect to mature B-, T- and NK-cells. The improved cancer cell line engraftment rate and tumor growth kinetics was novel and completely unexpected.

Methods of Preparing the Rat KO

The SCID rats may be prepared using a variety of gene editing techniques; for example, they may be prepared using zinc-finger nucleases, CRISPR/Cas, TALEN, such as XTN™. See, for example, U.S. Pat. Nos. 8,993,233, 8,795,965, 8,771,945, 8,889,356, 8,865,406, 8,999,641, 8,945,839, 8,932,814, 9,902,971, each of which is incorporated by reference for all purpose, and in particular for methods of gene editing. In certain aspects, the CRISPR/Cas system can employ a Cas9 nuclease, which in some instances, is codon-optimized for the desired cell type in which it is to be expressed. The system further employs a fused crRNA-tracrRNA construct that functions with the codon-optimized Cas9. This single RNA is often referred to as a guide RNA or gRNA. Within a gRNA, the crRNA portion is identified as a target sequence for the given recognition site and the tracrRNA is often referred to as the ‘scaffold’. This system has been shown to function in a variety of eukaryotic and prokaryotic cells. Briefly, a short DNA fragment containing the target sequence is inserted into a guide RNA expression plasmid. The gRNA expression plasmid comprises the target sequence (in some embodiments around 20 nucleotides), a form of the tracrRNA sequence (the scaffold) as well as a suitable promoter that is active in the cell and necessary elements for proper processing in eukaryotic cells. The system may rely on complementary oligonucleotides that are annealed to form a double stranded DNA and then cloned into the gRNA expression plasmid. The gRNA expression cassette and the Cas9 expression cassette are then introduced into the cell. See, for example, Mali P et al. (2013) Science 2013 Feb. 15; 339 (6121):823-6; Jinek M et al. Science 2012 Aug. 17; 337(6096):816-21; Hwang W Y et al. Nat Biotechnol 2013 March; 31(3):227-9; Jiang W et al. Nat Biotechnol 2013 March; 31(3):233-9; and, Cong L et al. Science 2013 Feb. 15; 339(6121):819-23, each of which is herein incorporated by reference.

After preparing of a single knock out, the founders may be mated to produce a double knockout SCID rat. In certain aspects, the IL2rg and/or RAG2 genes contain a mutation that eliminates expression or reduces expression such that the animal does not produce an amount of protein adequate to carry out normal function. In certain aspects, the mutation is a deletion. In preferred aspects, the SCID rat is homozygous for a Rag2 deletion and an IL2rg deletion (i.e., Rag2^{-/-}, IL2rg^{-/-}). In certain embodiments, the sequence deleted from the Rag2 gene comprises SEQ ID NO:1 and the sequence deleted from the IL2rg sequence comprises SEQ ID NO:2. In other aspects, the sequence deleted from the Rag2 gene consists of SEQ ID NO:1 and the sequence deleted from the IL2rg sequence consists of SEQ ID NO:2. In particular aspects, no other genes are mutated compared to the wild-type animal.

Analysis of whole blood demonstrated that while a wild-type rat has about 37.4% CD4+, about 36.6% CD8+, and about 3.5% CD4+/CD8+ in the circulating lymphocytes. In contrast, the SCID rat has depleted or substantially depleted mature CD4+, CD8+ and NK cells compared to the wild-type. Thus, in preferred aspects, the CD4+ cells are present at about 1.0% to about 2.0%, for example about 1.5%, compared to about 37.4% percent in a wild-type animal. Thus, in particular aspects, depletion of CD4+ T cells may be at least 80%, at least 85% or at least 90%. In certain aspects, depletion of CD8+ T cells may be at least 80%, at least 85% or at least 90%.

In other aspects, CD8+ cells are present at about 4-6%, for example about 5%, compared to about 36.6% in the wild-type animal. The portion of CD4+/CD8+ cells in the SCID rat double knock out may be about 0.5% to about 1.5%; for example, about 1.2%, in contrast to about 3.5% in the while type animal. In yet other preferred aspects, the ILR2g and Rag2 KO rat is devoid of mature B cells in the spleen and/or in the circulation, using cell surface markers CD45 or IgM for detection.

In particular aspects, the NK cells are also substantially depleted or depleted. For example, the circulating NK cells, as measured by CD161 antibody, may be less than 1.0%; for example, about 0.5% or about 0.1% to about 0.9%, as a percentage of PBMCs. In contrast, a wild-type rat has circulating NK cells about 10%. Thus, in particular aspects, depletion of circulating NK cells is at least 85%, at least 90% or at least 95%.

In preferred aspects, the rat is a Sprague Dawley rat. In other aspects, the rat is a Long Evans rat, a Wistar Kyoto rat, a Fischer 344 rat or a Brown Norway rat.

SCID Rat Xenograft Model

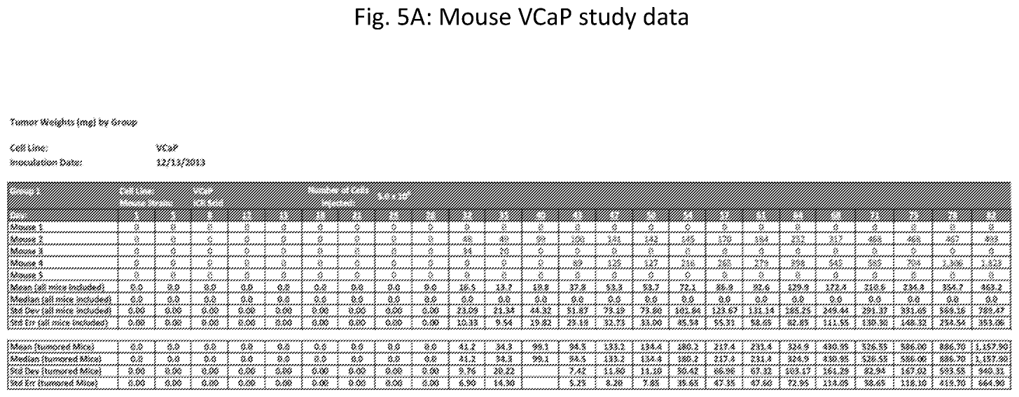

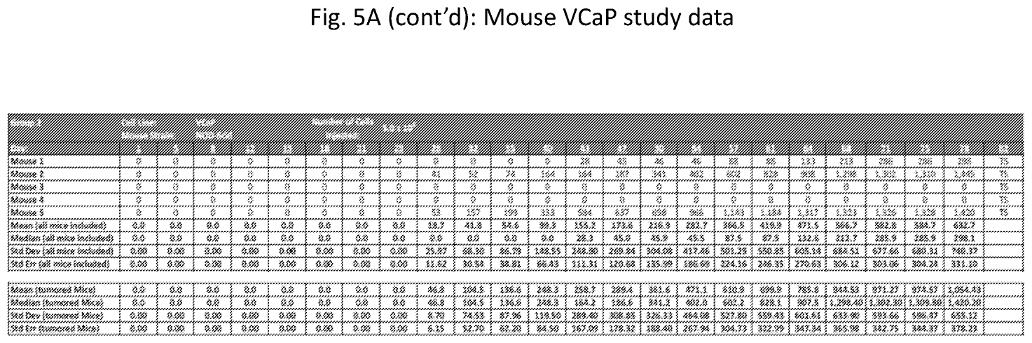

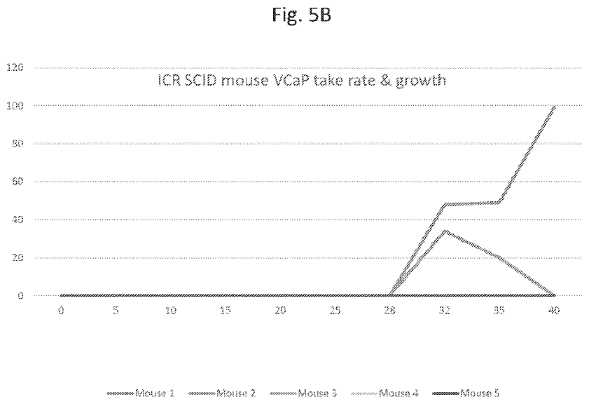

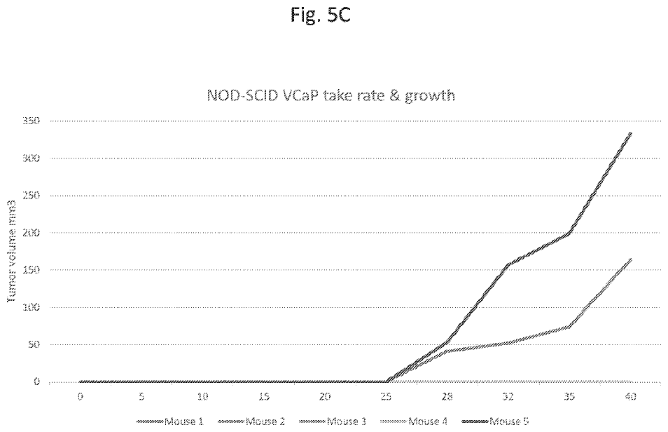

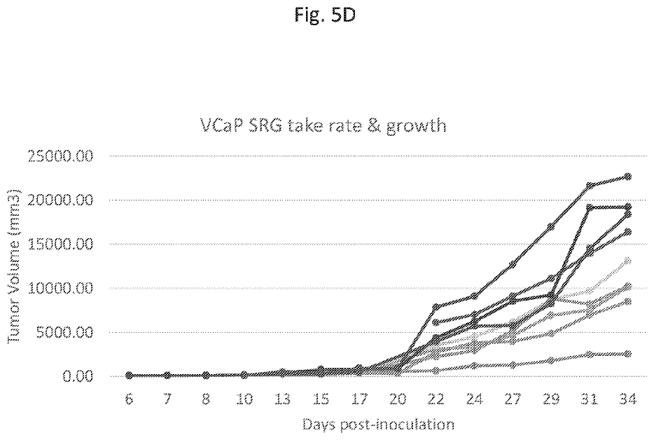

The Rag2^{-/-}, IL2rg^{-/-}SCID rat is particularly useful as a model because of the unexpectedly superior growth rates compared to similar mouse models. In the mouse 4 to 8 months are required for establishment of a PDX model with enough animals for a preclinical study. In contrast, the Rag2^{-/-},IL2rg^{-/-} shows xenograft tumor growth rates that makes the rat ready for screening drugs at a much earlier timepoint than would have been expected, which provides an excellent commercial advantage. Thus, in contrast to the rat models disclosed herein, the corresponding mouse does not effectively grow xenograft tumors. The contrast is illustrated in FIG. 5. FIGS. 5B and 5C show that only VCap cells grew in 2 of 5 mice tested (i.e., the take rate was only 40%) in two separate mouse models, the ICR SCID mouse and the NOD SCID mouse. Moreover, in one of the ICR SCID models where the tumor did take, it regressed completely. FIG. 5D illustrates the consistent take rate and growth in the rat model. In another study, the take rate was about 85% for the rat SCID model and about 20% in mice for the VCap cell line. The mouse take rate is thus both low, and variable, which, combined with the tendency for spontaneous regression, means that the mouse models are inadequate.

In contrast, the SCID rats have better take rates than the equivalent SCID mouse for cancer xenografts. In particular aspects, the rat take rate is about 30%, about 40%, about 50%, about 70%, about 80%, about 90%, or about 100%, whereas the corresponding mouse take rate is lower by about 10 points, about 20 points, about 30 points, about 40 points, about 50 points, about 60 points, about 70 points, or about 80 points. Thus, for example, where a rat xenograft has a take rate of about 90%, the corresponding mouse xenograft take rate may be about 70 points lower; i.e. about 20%. The take rate is measured at a suitable time-point; for example, at 10 days post-implantation.

In other aspects, the SCID rats have better growth kinetics for the cancer xenografts. For example, the tumor growth rate in the rat is about 5%, about 10%, about 20%, about 50%, about 75%, about 100%, 200%, 500%, 1000% or more faster than a corresponding SCID mouse. Percentage tumor volume is calculated as: % Group Mean Change=((X−X0)/X0)*100 where X=current mean, X₀=Initial mean. In particular examples, the % tumor volume group mean for IL2rg;Rag2 KO rat and corresponding SCID mouse over comparable time points was for HCT116 1,971% and 315% respectively, and for VCaP 2,133% and 530%, respectively.

The enhanced tumor growth kinetics results in a rat having a xenograft tumor with a tumor volume in a range of about 20,000 to about 40,000 mm³, or about 1000 mm³ to about 10,000 mm³ or about 100 mm³ to 1,000 mm³ or about 10,000 mm³ to 20,000 mm³ In particular examples, the range is about 700 to about 25,000 mm³ for VCaP about 500 to about 10,000 mm³ for H358, about 200 to about 6,000 mm³ for HCT116 and about 2,000 to about 20,000 mm³ for OCI-AML2. While the tumor volume varies with the xenograft, the permissive growth environment of the rat insures dramatically improved results compared to a corresponding mouse.

Compared to the corresponding SCID mouse xenograft model this means that time to establish the SCID rat xenograft model—for example, for use in drug efficacy testing—is reduced. For example, the time to establish a SCID rat xenograft model may be about 1 month less than the mouse, about 2 months less than the mouse, about 3 months less than the mouse, about 4 months less than the mouse, about 5 months less than the mouse, or about 6 months less than the corresponding SCID mouse xenograft model.

Thus, in particular aspects, the rat xenograft models disclosed herein are ready for performing assays (e.g. a drug efficacy assay) at the passage 1 (P1) stage or P2 stage. In other aspects, where a xenograft is particularly challenging, the rat model may not be ripe for assays until P3, P4, P5 or later stages. The P0 animal is the animal that first receives the xenograft. The P1 animal receives the xenograft from the P0 animal, and so on. In each case, however, the rat model is available earlier than the corresponding mouse is ready. For example, the rat xenograft model is ready at least 1 passage earlier, at least 2 passages earlier, or at least 3 passages earlier, than the corresponding mouse.

When the xenograft is from a patient (i.e., a PDX), the biopsy tissue may be removed and preserved in typical cell culture medium and is then cut into portions which are then introduced into the SCID rat. In certain aspects, the biopsy portion introduced is substantially cube-like and has dimensions of 2 mm along each side.

Moreover, the Rag2^{-/-}, IL2rg^{-/-}SCID rat is superior to the single Rag2^{-/-} rat (referred to herein as SDR). In an experiment using the OCI-AML2 cell line as a xenograft, the Rag2^{-/-}, IL2rg^{-/-} SCID rat showed a 100% take rate, whereas the Rag2^{-/-} rat showed a 0% take rate.

Xenografts

A variety of xenografts may be used for growth in the Rag2^{-/-}, IL2rg^{-/-} SCID rat. In some aspects, the xenograft is produced using established cancer cell lines. In other aspects, an implant may be prepared from a tumor sample taken from a patient during biopsy. The ability to grow such biopsy tissue (See FIG. 6) is particular advantageous because such patient-derived xenografts (PDX) closely model the response of a tumor in the patient. The primary tumor may be from subjects having a variety of cancers; for example, the primary implant may be from a breast cancer, a prostate cancer, a melanoma, a colon cancer, a lung cancer, a lymphoma, a pancreatic cancer, an endometrial cancer, a thyroid cancer, an ovarian cancer or a bladder cancer. In a preferred example, the biopsy tissue is from a subject diagnosed as having an ovarian cancer. In another preferred example, the biopsy tissue is from a subject diagnosed as having a non-small cell lung cancer (NSCLC).

Advantageously, the NSCLC PDX model exhibits particular phenotypes useful for drug testing and for research. The size of the NSCLC PDX tumor can exceed 28 mm, with the volume over 32000 mm³ and we have found that under circumstances where the tumor length is greater than 25 mm, the core of the xenograft contains necrotic tissue. At later stages of development, the inner core of the tumor will be necrotic tissue, surrounded by a shell of quiescent cells (which often proliferate subsequent to therapy), and an outermost layer of live, proliferating cells. While most anti-cancer therapies focus on the proliferating cells, the rat models disclosed provide opportunities to assess the role of quiescent cells and the necrotic core in tumor pathology and in response to drug treatment, as well as the proliferating cells. The ability of the rat model to support tumor growth that accurately mimics naturally-occurring tumors reinforces the excellent utility of the rat model.

In particular aspects, the xenograft may be grown using cancer cell line; typically, a human cell line. The cell line may be from a variety of cancer types; for example, the cell line may be a breast cancer cell line, a prostate cancer cell line, a melanoma cell line, a colon cancer cell line, a lung cancer cell line, a lymphoma cell line, a pancreatic cancer cell line, an endometrial cancer cell line, a thyroid cancer cell line, an ovarian cancer cell line, or a bladder cancer cell line.

In some aspects, the breast cancer cell line is selected from the group consisting of MCF7, BT-20, MDA-MB-231, MDA-MB-453, and BT474. In some aspects, the colon cancer cell line is selected from SW-620, HCT116, SW-480, HT-29, and CT-26. In some aspects, the prostate cancer cell line is selected from the group consisting of VCaP, LNCaP, PC-3, 22Rv1, and DU-145. In some aspects, the leukemia cell line is selected from the group consisting of Jurkat, MV4-11, HL-60, THP-1, and REH. In some aspects, the lung cancer cell line is selected from the group consisting of A549, Calu-6, H358, Calu-3, and KYSE-30. In some aspects, the bladder cancer cell line is selected from group consisting of 786-0, A498, SW 780, and A498. In some aspects, the ovarian cancer is selected from the group consisting of SK-0V-3, OVCAR-3, OVCAR-5, and A2780. In some aspects, the brain cancer cell line is selected from the group consisting of U251 and U87-MG. The hepatocellular cancer cell line HepG2 may be used. The cell line may be the pancreatic cell line MiaPaCa-2 or PANC-1. The melanoma cell line may be A375. In other aspects, the FaDu cell line, derived from a squamous cell carcinoma, may be used. In preferred aspects, the cell line is selected from the group consisting of VCaP, H358, and HCT-116. In particular aspects, the cell line is OCI-AML2.

The cell lines are grown using conventional cell culture approaches and then injected into the animals subcutaneously. Typically, the cells are injected with a component that mimics extracellular matrix. Suitable extracellular matrix mimics may contain one or more of laminin, entactin/nidogen, collagen and heparan sulfate proteoglycans, and also growth factors like TGF-beta and EGF. Commercially available options include Cultrex® BME3 (Trevigen® #3632-001-02), Geltrex® (Gibco™), and Matrigel® (Corning®).

The number of cells introduced into the rat to form the xenograft may vary. For example, the number of cells may be in a range from about 1×10⁶ to about 10×10⁶, about 1×10⁶ to about 5×10⁶, or about 5×10⁶ to about 10×10⁶. Counting of cell numbers may be performed by methods known in the art.

In certain aspects, the xenograft introduces a human immune system into the Rag2^{-/-}, IL2rg^{-/-} SCID rat. Thus, in certain aspects, the xenograft comprises, or consists of, peripheral blood mononuclear cells (PBMCs). Previous attempts to use mice with similar genetic defects failed because the animals developed signs of GvHD at a timepoint that was sufficiently early such that the model was not useful. In contrast, however, implanting PBMCs into a rat SCID model did not result in problematic onset of GvHD symptoms. Thus, in particular aspects, disclosed herein are SCID rats having a humanized immune system having delayed onset of GvHd symptoms. In particular aspects, GvHD does not appear until after 6 weeks post-implantation, after 7 weeks post-implantation, after 8 weeks post-implantation, after 9 weeks post-implantation, after 10 weeks post-implantation, or after 11 weeks post-implantation. Thus, in contrast to mice where GvHD onset is about 4-5 weeks, the disclosed Rag2^{-/-}, IL2rg^{-/-} SCID rat model provides greater utility for studies requiring analysis of the human immune system. In certain other aspects, the xenograft used to produce a humanized immune system may comprise or consist of human hematopoietic stems cells (HSC), including, for example CD34+ HSC cells.

The xenograft SCID rats containing humanized immune systems may be used in methods to assess various responses including tumor-immune system interactions, tumor immune system escape, and the therapeutic effect of immune system modulation on tumor growth.

In particular aspects, a xenograft tumor may be removed from the rat and introduced into a second animal type, where the second animal type differs from the first. In some aspects, the second animal type is a rat with a different genetic background. In other aspects, the second animal type is not a rat. For example, the second animal type may be a mouse, a dog, a rabbit, a hamster, a macaque, or a chimpanzee. In particular aspects, the mouse may be a knock-out mouse. In some cases, the knock-out mouse may be a comparable knock-out mouse; in other cases, the knock-out mouse may have different genes knocked out. Advantageously, the excellent xenograft growth in the rat provides a substantial amount of tumor tissue that can divided amongst multiple animals for performing assays. Such an approach offers particular economic value as performing assays in the mouse requires less materiel and mice are cheaper to use as a model generally. In particular aspects, the xenograft type is one that grows well when directly introduced into the second animal type; in other aspects, the xenograft type is one that does not grow well when directly introduced into the second animal but shows improved growth kinetics in the second animal type; for example, in a mouse following growth in the SCID rat.

Drug Efficacy Assays

The SCID rat knock-out models containing xenografts disclosed herein are particularly useful for drug efficacy studies. The route of administration of the drug may be subcutaneous, intraperitoneal, intravascular (intravenous and intra-arterial), intramuscular, topical, intradermal, oral, mucosal, or ocular. In particular aspects, administration is by tail vein injection.

A variety of pharmaceutical and biologic anti-cancer drugs may be tested. For example, the drug may be a PARP inhibitor such as Talazoparib (BMN-673), Veliparib, Olaparib, Rucaparib (AG014699, PF-01367338), Veliparib (ABT-888).CEP 9722[30], E7016, BGB-290. In certain aspects, the drug may be an antisense oligonucleotide, a microRNA, or an RNAi. In other aspects the drug may be designed as a cancer-specific drug. For example, enzalutamide is a suitable prostate cancer drug. In yet other aspects, the drug is a biologic such as an antibody, a CAR-T cell, or another cell-based therapy.

Efficacy of the drug may be determined by any suitable means. For example, in certain assays, the response to the drug may be measured related to impact on tumor growth compared to control. In other aspects, particular molecules produced by the tumor or cells expressing particular genes may also be monitored (for example, by PCR or by FACS analysis); for example, where the xenograft is derived from a prostate cancer, either by biopsy or by using cancer cell line such as VCap, the amount of Prostate Serum Antigen (PSA) produced by the xenograft may be monitored.

It is a particular advantage of the rat xenograft models that the excellent take rate and rapid tumor growth allow drug studies to be initiated soon after xenograft implantation. In particular aspects, the rat is ready for drug studies (e.g. a screen or assay) at 1, 2, 3, 4, 5, or 6 months post-implantation of the xenograft.

In other aspects, the xenograft tumor may be removed from the rat and used to perform assays in vitro. Advantageously, the high take rate and growth kinetics mean that, compared to an equivalent mouse, about 10-fold more cellular tissue is available for such assays. Thus, in particular aspects, the methods disclosed herein include growing a xenograft in a SCID rat model, harvesting cells of the xenograft, and using the harvested cells in an assay. Because the xenografts grow well, cell harvesting may take place about 1, 2, 3, 4, 5, or 6 months post-implantation of the xenograft.

The SCID rat models having a humanized human system may be used in a variety of ways. For example, they may be used to determine the impact of cell therapies such as chimeric antigen receptor (CAR)-T cells. In other aspects, they may be used to assess blockade of checkpoint proteins; for example, they may use to test efficacy of drugs that modulate the activity of programmed cell death protein 1 (PD-1), PD-L1, or CTLA-4. For example, new antibodies against checkpoint inhibitors may be assayed.

### EXAMPLES

### Example 1

Generation of Rag2- and Rag2/IL2RG-Knockout Sprague Dawley Rats

For the single knockout, the Rag2 locus was targeted using XTN™ technology in spermatogonial stem cells (SSCs). Pooled SSCs were transplanted into DAZL-deficient sterile males and mated with wild-type Sprague Dawley rats. DNA was isolated from offspring and a male with a 27 bp deletion was detected.

For the double-knockout, the Rag2 and Il2rg loci were targeted using CRISPR. CRISPR based targeted nuclease reagents targeting the Rag2 and Il2rg genes were microinjected into Sprague Dawley embryos at the 2-cell stage. A total of 314 embryos were injected, of which 187 were successfully transferred into pseudopregnant surrogates. 32 animals were born of which, 9 animals carried at least one mutated allele verified by targeted sequence analysis. These founders were interbred to create the Rag2.I2rg double knockout animal (Rag2^{-/-}, IL2rg^{-/-}), which contains an 8 bp homozygous deletion in the Rag2 gene (atatggcc; SEQ ID NO:1) and 16 bp homozygous deletion (gagaatctaggctcat; SEQ ID NO:2) in the Il2rg gene.

FACS analysis of immune cells: To detect T, B, and NK cells, flow cytometric analysis was performed on splenocytes and thymocytes. Cells were stained with fluorophore-labeled antibodies at a final concentration of 25 μg/ml in 20 μl volume for 20 minutes. Antibodies used were Goat anti-rat IgM-APC (Stem Cell Technologies #10215), PE Mouse anti-rat IgM (BD #553888), FITC Mouse anti-rat CD45R (BD #561876), APC Mouse anti-rat CD45R (BD #554881), FITC Mouse Anti-Rat CD8b (BD #554973), PE Mouse anti-rat CD8b (BD #554857) APC Mouse Anti-Rat CD4 (BD #550057), FITC Mouse Anti-Rat CD161a (BD #561781), APC Mouse anti-rat CD161a (BD #555009).

Transplantation of human cancer cell lines: 1 million cells (U87MG human glioblastoma) or 1, 5, or 10 million cells (H358 human non-small cell lung cancer cells) were mixed with Geltrex® 1:1 and transplanted subcutaneously in the hind flank. Tumors were measured three times weekly and recorded in StudyLog to determine tumor growth kinetics. Animals were euthanized before the tumors reached humane endpoints.

Immunohistochemistry for human proteins: Tumors were excised and fixed in 10% NBF. Standard Sum sections were collected and human cells were visualized by staining with an antibody that recognizes a protein found in all human mitochondria (mouse anti-human mitochondria antibody, clone 113-1; EMD Millipore #1273) at 1:250.

### Example 2

Improved Human Non-Small Cell Lung Cancer (NSCLC) Tumor Engraftment and Kinetics in Rag2 KO Rats

The Rag2 knockout, demonstrated improved tumor growth kinetics and engraftment rate for H358 xenografts. A KRAS mutant non-small cell lung cancer (NSCLC) cell line H358 was implanted into Rag2 KO rats subcutaneously. 1, 5, or 10 million cells (H358 human non-small cell lung cancer cells) were mixed with Geltrex® 1:1 and transplanted subcutaneously in the hind flank. Tumors were measured three times weekly and recorded in StudyLog to determine tumor growth kinetics.

The tumor growth was faster and more consistent when compared to NSG and Nude Mice (FIG. 4). A 100% tumor engraftment rate observed was observed in the Rag2 KO rats, compared to less than 20% successful engraftment rate in immunodeficient mice. Tumor kinetics were also much better in the Rag2 KO rat: the growth curve of the tumor within a treatment group were much closer to each other than what was observed in the mouse. Corresponding with the higher engraftment efficiency, the tumors grew much faster in the Rag2 KO rat compared to the NSG mouse even in rats implanted with only 1 million cells (as opposed to 10 million in NSG mouse). These data show that the NSCLC KRAS mutant cell line H358 has 100% survival when transplanted in the Rag2 KO rat and shows faster and more uniform growth kinetics compared to growth in the Nude and NSG™ mouse.

### Example 3

Improved Human Prostate Cancer Tumor Engraftment and Kinetics in Rag2^{-/-}, IL2rg^{-/-} Rats

In a study for the evaluation of growth kinetics for VCaP human prostate tumor xenograft model using Nod SCID and ICR SCID, the tumor engraftment rate and growth kinetics were poor. In both models 3 of 5 mice showed tumor engraftment but not until at least day 28 and 32 for NOD and ICR SCID mice respectively (FIG. 5A-5C). The tumor kinetics were slow and very inconsistent, some mice showing tumor growth at day 28 while others not until day 43. Under the tumor engraftment and growth kinetics using SCID mice conducting an efficacy study was very difficult and results from such a study could not be relied on.

However, when the same VCap human prostate tumor xenograft model was implanted on both flanks of ILR2g and Rag2 KO rats, we achieved tumor growth within less than 14 days. For transplantation, 10×10⁶ VCaP cells for each animal were resuspended in 250 μL sterile 1×PBS (Gibco #14190-144). Immediately prior to injection, 250 μl 10 mg/ml Cultrex BME3 (Trevigen #3632-001-02) was added to the cell suspension for a final Cultrex concentration of 5 mg/ml. The cell/Cultrex suspension was injected subcutaneously into the hindflank. Tumor diameter was measured using digital calipers (Fisher #14-648-17) 3 times a week. Tumor volume was calculated as (L×W²)/2, where width and length were measured at the longest edges

FIG. 5D illustrates the tumor kinetics in the ILR2g and Rag2 KO SCID rat. Each line represents tumor growth in an individual ILR2g and Rag2 KO rat. Unlike the equivalent knockout mouse, the rat illustrates a 100% take-rate and, moreover, VCaP tumors in the SCID rat are at or above 20,000 mm³ by around 4-5 weeks post-inoculation.

### Example 3

Enhanced Growth with HCT-116 Xenograft Model in the ILR2g and Rag2 KO SCID Rat Vs. NSG Mouse.

We also assessed the ability of the HCT-116 xenograft to form tumor xenografts.

For transplantation, 2×10⁶ HCT-116 cells for each animal (NSG mice and ILR2g and Rag2 KO rats) were resuspended in 250 μl sterile 1×PBS (Gibco #14190-144). Immediately prior to injection, 250 μl 10 mg/ml Cultrex BME3 (Trevigen #3632-001-02) was added to the cell suspension for a final Cultrex concentration of 5 mg/ml. The cell/Cultrex suspension was injected subcutaneously into the hindflank. Tumor diameter was measured using digital calipers (Fisher #14-648-17) 3 times a week. Tumor volume was calculated as (L×W²)/2, where width and length were measured at the longest edges.

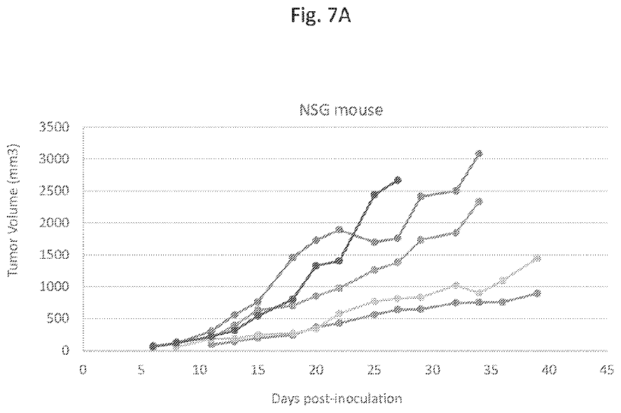

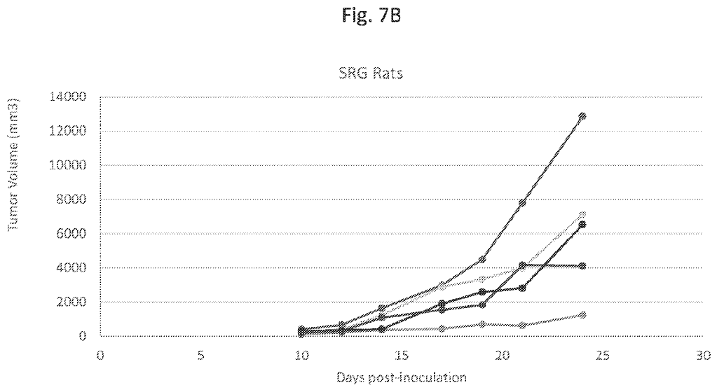

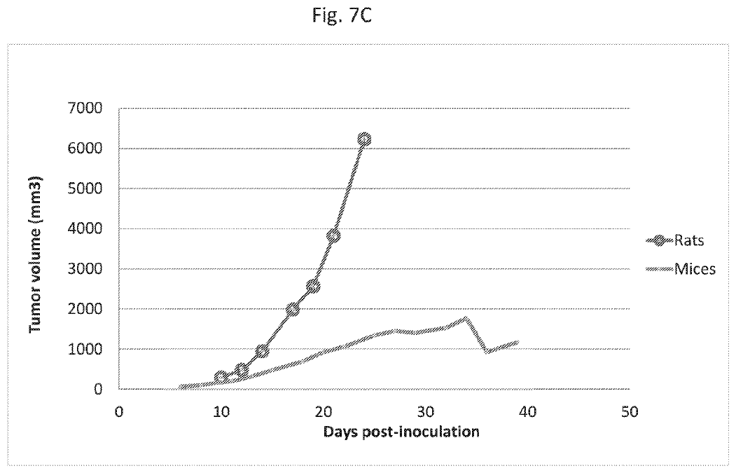

FIG. 7A shows tumor kinetics in 5 NSG mice. Each line represents an individual mouse. FIG. 7B shows tumor kinetics in 5 ILR2g and Rag2 KO rats, where each line represents an individual rat. FIG. 7C provides a comparison of growth kinetics in the ILR2g and Rag2 KO rat vs. NSG mouse. Each line represents the average tumor volume in 5 animals for each species. This comparison shows that the growth kinetics of the xenograft are remarkably different. Even accounting for the difference in size between a rat and a mouse, these data—with equal numbers of cells administered to the rat and the mouse—illustrate that the growth rate in the rat advantageously provides larger tumor size at earlier timepoints.

### Example 4

Humanization of Immune System

We prepared Rag2;Il2rg KO rats as described above. We then injected 20×10⁶ viable human PBMCs in 500 μl PBS into the tail vein of 3 male and 3 female rats at 8-10 weeks of age. Peripheral blood was analyzed for the presence of human CD45+, CD3+, CD4+, CD8+ and CD20+ cells at 3, 7, 14, 28, 54, and 70 days post-injection.

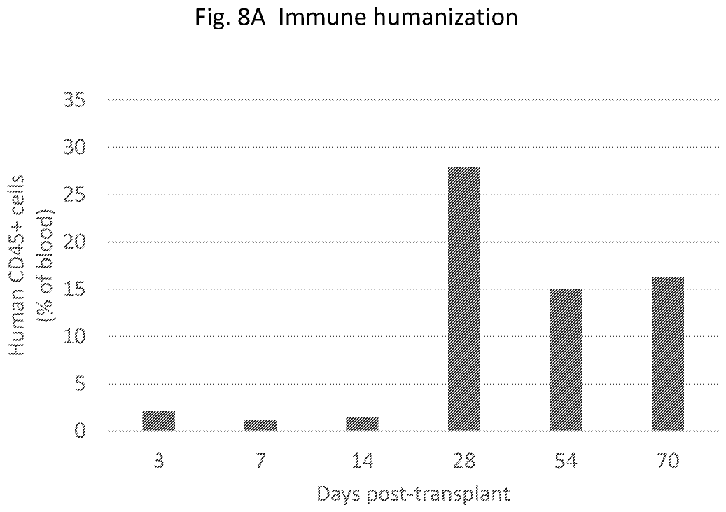

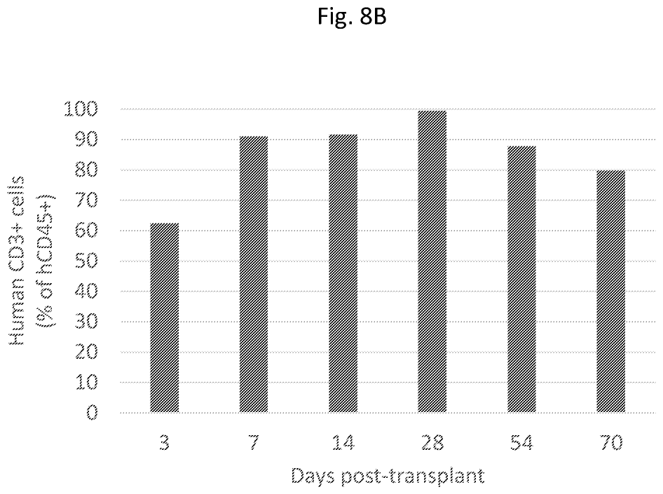

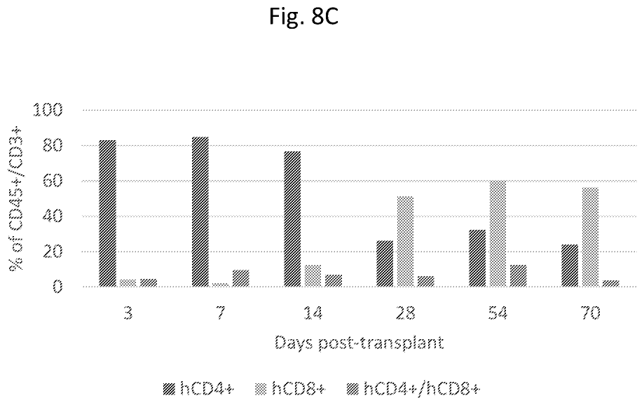

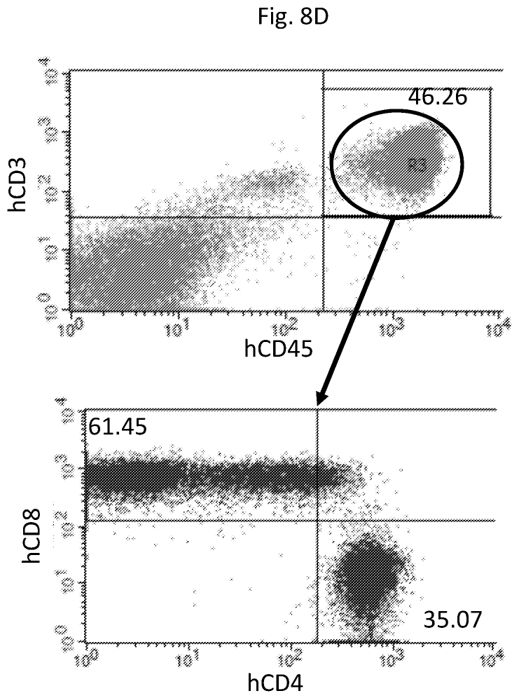

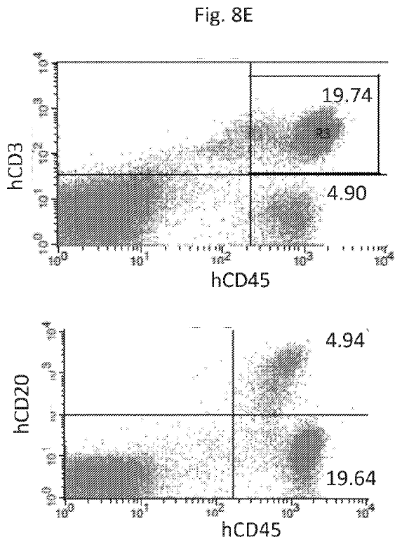

FIG. 8A shows that at 4 weeks post-transplant, recipients had an average of 29% circulating human CD45+ cells. At 10 weeks post-transplant, recipients had an average of 17% human CD45+ cells. FIG. 8B shows the population of CD 80% of CD45+ cells which were also CD3+ throughout the study. Before 28 days, there is a small population of hCD45+ cells that were not hCD3+, but nearly all hCD45+ cells were CD3+ by 28 days. At 70 days, 80% of CD45+ cells were CD3+ T cells.

FIG. 8C shows the distribution of CD4+, CD8+, and CD4+/CD8+ cells of the hCD45+/hCD3+ population in peripheral blood.

FIG. 8D shows FACS analysis of peripheral blood at 70 days post-transplant. Top plot shows CD45+/CD3+ cells. Bottom plot shows the breakdown of hCD3+ cells that are CD4+, CD8+, and CD4+/CD8+. This particular recipient had 46% circulating human CD45+ cells and remained healthy, with no signs of GvHD.

FIG. 8E shows a FACS analysis of an individual rat's peripheral blood at 70 days post-transplant. Top plot shows CD45+/CD3+ cells. There is a population of hCD45+/hCD3− cells, which appear to be hCD20+ (bottom plot). While only a subset of PBMC-injected rats had circulating human CD20+ cells, all rats who had successful engraftment had a large proportion of hCD20+ cells in the spleen.

After tail vein injection of 50 million human PBMCs, we were able to detect human CD45+ in the circulating blood of 66% of recipient rats. As expected, the majority of these CD45+ cells were also human CD3+. By 28 days post-transplant, nearly 100% of hCD45+ cells detected in the peripheral blood were CD3+. Interestingly, a subset of successfully engrafted rats had a small percentage of human CD45+ cells that were CD3−, which we later determined were human CD20+, a marker of B cells.

In the SCID mice transplanted with human PBMCs, there are no reports of circulating human B cells, though the spleens of these mice contain a significant population of human CD20 cells. In contrast to the mouse, we determined that the ILR2g and Rag2 KO rats contained circulating B cells based on the presence of circulating CD20+ cells.

We also determined that the ILR2g and Rag2 KO SCID rats displayed graft versus host disease (GvHD), marked by rapid body weight loss, loss of body condition, and lymphocyte infiltration in peripheral tissues. While GvHD is a hallmark of successful engraftment of human PBMCs, due to the presence of mature human T cells which eventually attack the recipient, it is notable that the ILR2g and Rag2 KO rats with successful engraftment did not exhibit symptoms until 8 weeks post-transplantation or later, a later onset compared to PBMC-engrafted mice.

### Example 5

Lung Cancer Primary Tumor Implant Xenograft

We compared the ability of primary tumors from patients to grow in NSG mice and ILR2g and Rag2 KO rats. NSG mice (NOD.Cg-Prkdc^{scid} Il2rg^{tm1Wjl}/SzJ) were obtained as described above. ILR2g and Rag2 KO rats were prepared as described in Example 1. We obtained tissue to use as a primary cell implant from a patient diagnosed as having NSCLC. We obtained the biopsy sample in media: DMEM (Gibco #11965092), 10% FBS (Atlanta Biologicals #S12452), 1% Penicillin/Streptomycin (Gibco #15070063), 1% Amphotericin B (Gibco #15290026). The biopsy tissue was rinsed in PBS. The biopsy was cut into approximately cube-like structures with 2 mm side length for implantation. These pieces were kept in media on ice until ready to transplant. Prior to implantation the tissue piece is rinsed in sterile PBS. A sterile forcep was used to place it into an autoclaved trocar and the tissue (in the trocar) was kept in the dish of PBS to prevent it from drying out.

A small (1 mm) incision was made just below the left shoulder blade with sterile scissors or a 16 G needle. Tenting the skin at the incision using sterile forceps, the trocar was placed into the incision. The trocar was gently pushed and guided through the subcutaneous (SQ) space until the tip reached the left dorsal hind flank. The trocar plunger was then inserted to push the biopsy piece into the SQ space.

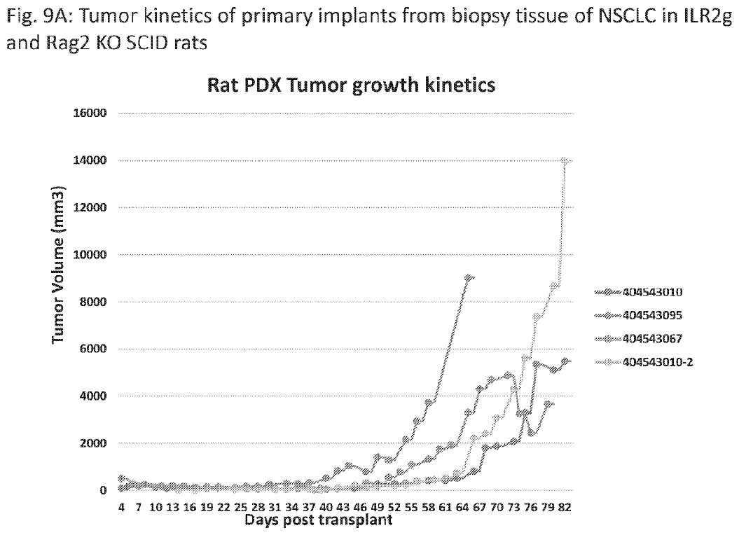

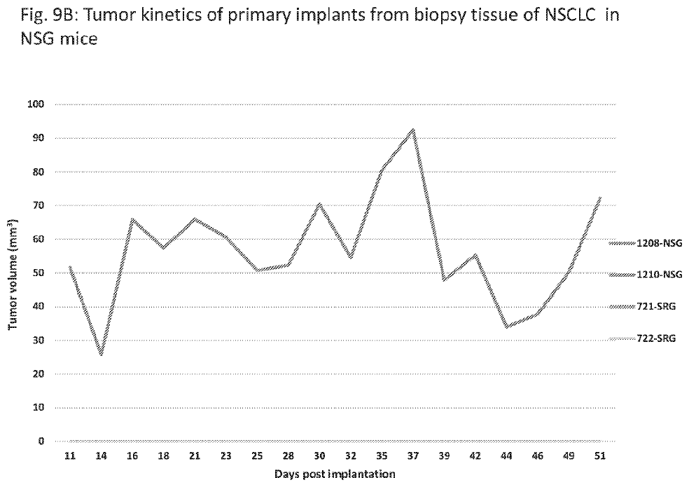

After introducing the tissue into ILR2g and Rag2 KO rats and into NSG mice we compared the ability of those two models to grow the primary implant. FIG. 9A shows that the primary implant grew extremely well. After about 40 days tumors grew in each of the rats. The tumor volume rose to about 4000 mm³ in each rat. Note that one rat shows a temporary dip in tumor size. In contrast, the mouse data shows poor growth. See FIG. 9B, showing only one engraftment (1208-NSG) that showed poor growth, around 60-80 mm³ by Day 51.

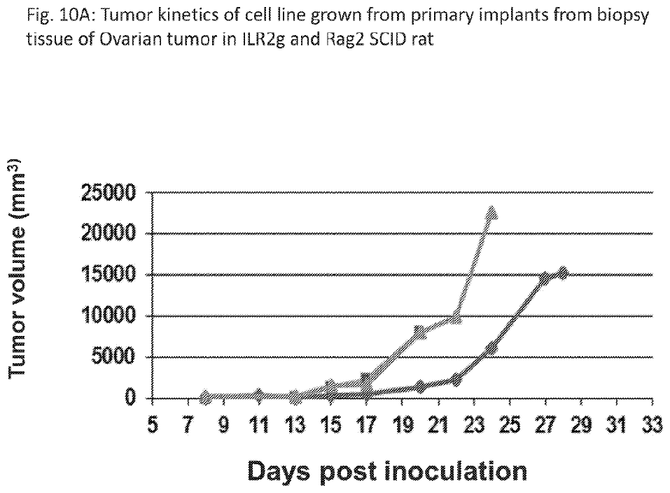

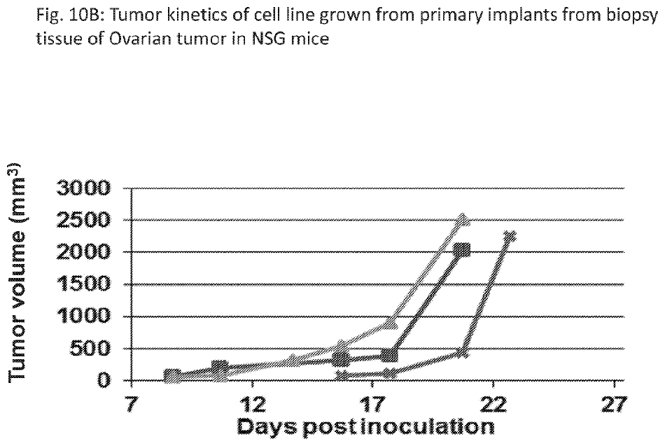

We obtained essentially identically superior results with an ovarian primary tumor. Compare FIGS. 10A and 10B.

### INCORPORATION BY REFERENCE

All patents, patent applications, and published scientific papers are incorporated by reference for all purposes.

1. A method of screening a drug for treating a tumor comprising
(a) administering the drug to a Severe Combined Immune Deficiency (SCID) rat having a xenograft tumor; wherein the SCID rat is a knockout rat comprising one or more genetic mutations that result in substantially depleted B-cells, T-cells and NK-cells; and
(b) determining the effect of the drug on the tumor.

2. The method of claim 1 wherein the SCID rat is an P1 rat or a P2 rat.

3. The method of claim 1 wherein the SCID rat has a percentage take rate at least 10 points higher than a corresponding SCID mouse.

4. The method of claim 1 wherein the SCID rat has a tumor growth rate at least 5% than a corresponding SCID mouse.

5. The method of claim 1 wherein the SCID rat contains a deletion in the IL2Rg and the Rag2 genes.

6. The method of claim 5 wherein the deletion in the Rag2 gene comprises SEQ ID NO: 1 and the deletion in the IL2Rg gene comprises SEQ ID NO:2.

7. The method of claim 1 wherein the screen is performed around 2 months post xenograft implantation.

8. The method of claim 1 wherein the average xenograft tumor volume is 6,000 to 40,000 mm³.

9. The method of claim 1 wherein the xenograft is patient-derived xenograft (PDX) containing primary cells from tumor from a subject.

10. The method of claim 9 wherein the tumor is selected from a breast cancer, a prostate cancer, a melanoma, a colon cancer, a lung cancer, a lymphoma, a pancreatic cancer, an endometrial cancer, a thyroid cancer, an ovarian cancer, and a bladder cancer.

11. The method of claim 9 wherein the tumor is from a subject having a non-small cell lung cancer (NSCLC) or an ovarian cancer.

12. The method of claim 1 wherein the xenograft contains cells from a cancer cell line.

13. The method of claim 12 wherein the cancer cell line is a breast cancer cell line, a prostate cancer cell line, a melanoma cell line, a colon cancer cell line, a lung cancer cell line, a lymphoma cell line, a pancreatic cancer cell line, an endometrial cancer cell line, a thyroid cancer cell line, an ovarian cancer cell line, or a bladder cancer cell line.

14. The method of claim 12 wherein the cell line is selected from the group consisting of VCaP, H358, OV81, and HCT-116.

15. A Severe Combined Immune Deficiency (SCID) rat having a xenograft tumor, wherein the SCID rat is suitable for a drug screening assay; and wherein the SCID rat is a knockout rat comprising substantially depleted mature B-cells, T-cells and NK-cells.

16. The SCID rat of claim 15 wherein the xenograft tumor volume is about 6,000 to 40,000 mm³ at 30-60 days post-implantation.

17. The SCID rat of claim 15 wherein the knockout rat has a deletion in the Rag2 gene and the IL2Rg gene.

18. The SCID rat of claim 17 wherein the deletion in the Rag2 gene comprises SEQ ID NO: 1 and the deletion in the IL2Rg gene comprises SEQ ID NO:2.

19. The SCID rat of claim 15 wherein the SCID rat has a percentage take rate at least 10 points higher than a corresponding SCID mouse.

20. The SCID rat of claim 15 wherein the SCID rat has a tumor growth rate at least 5% higher than a corresponding SCID mouse.

21. The SCID rat of claim 15 wherein the rat is a Sprague Dawley rat.

22. A method of performing a drug efficacy study, comprising
(i) introducing a xenograft into a Severe Combined Immune Deficiency (SCID) rat; wherein the SCID rat comprises substantially depleted mature B-cells, T-cells and NK-cells; and wherein the xenograft is a patent-derived xenograft; and
(ii) administering the drug to the SCID rat, wherein the SCID rat is a P1 or P2 passage rat.

23. The method of claim 22 wherein the SCID rat is homozygous for deletions in the IL2Rg gene and the Rag2 gene.

24. The method of claim 22 and wherein the tumor cells are introduced in the presence of an extracellular matrix mimic.

25. The method of claim 22 wherein the SCID rat has a deletion in the Rag2 gene and the IL2Rg gene.

26. The method of claim 25 wherein the deletion in the Rag2 gene comprises SEQ ID NO: 1 and the deletion in the IL2Rg gene comprises SEQ ID NO:2.

27. The method of claim 22, wherein the SCID rat is a P1 passage rat.

28. The method of claim 22, wherein the SCID rat exhibits at least a 5% greater mean tumor volume (TV) growth than a comparable SCID mouse, wherein the comparison of tumor volume is performed when the SCID rat is at least 1 fewer passage than the mouse.

29. The method of claim 28 wherein the SCID rat is a P1 passage SCID rat and the SCID mouse is a P2 passage SCID mouse.

30. The method of claim 22 wherein the patient-derived xenograft is from an ovarian tumor or a non-small cell lung cancer (NSCLC).
